(19) United States
(12) Patent Application Publication
(10) Pub. No.: US 2008/0267688 A1
(43) Pub. Date: Oct. 30, 2008

(54) Apparatus and a Method For Cleaning a Channel in a Medical Instrument

(75) Inventor: Tommy Busted, Copenhagen (DK)

Correspondence Address: GIFFORD, KRASS, SPRINKLE,ANDERSON & CITKOWSKI, P.C, PO BOX 7021, TROY, MI, 48007-7021, US

(73) Assignee: Bat Holding ApS

(21) Appl. No.: 12/095,249

(22) PCT Filed: Nov. 29, 2006
(86) PCT No.: PCT/DK06/00671

§ 371 Date: Jul. 7, 2008

(30) Foreign Application Priority Data
Nov. 29, 2005 (DK) PA 2005 01683

(51) Int. Cl. A61B 1/12

(52) U.S. Cl. 401/190

(57) ABSTRACT

The present invention concerns a device and a method for cleaning a channel of a medical instrument, such as an endoscope, by discharging vaporised cleaning fluid and subsequently pressurised gas into the channel.

The present invention relates to a device for cleaning a channel in a medical instrument, said device comprising an elongated tubular member defining a flow passage having a first end and second end. The invention further relates to an apparatus and a method for cleaning a channel in a medical instrument.

A method and apparatus of this kind is known from U.S. Pat. No. 6,047,431.

An endoscope or other medical instruments are often provided with one or more biopsy channels. The medical instrument permits minimally invasive medical examinations of internal body tissues and are provided with channels for e.g. biopsies of the tissue, etc. After use, the medical instrument must be cleaned. The channels and passageways of the medical instrument may prove particularly difficult to clean and for this purpose there are provided various cleaning devices.

The cleaning devices typically comprise a small brush on a flexible shaft. As disclosed in U.S. Pat. No. 6,047,431, the shaft may be motorised and a cleaning fluid is passed through the tubular flexible shaft to the outlet next to the brush. The brush may be rotated and moved up and down the channel while the cleaning fluid is expelled for cleaning the channel.

In U.S. Pat. No. 5,240,675, an elongated cleaning member is described for cleaning a channel in an endoscope, where a vibrator mechanism is used together with a brush at the distal end of the elongated member. Furthermore, the cleaning member may be provided with a radiation source for sterilising the channel.

The medical instruments are fragile and therefore one must be careful when cleaning the instrument and in particular when cleaning the channels. The brushes must be resilient and sufficiently smooth in order to avoid damaging the internal walls of the channels and the brushes must be dimensioned according to the diameter of the individual channels.

The drawback of the channel cleaning methods for medical instruments, such as ultra sound transducers or endoscopes, is that there is a risk that the channels are not properly cleaned as the connectors easily fall off. Moreover, impurities may form as residues on the inside walls of the channels and by a mechanical brush cleaning, this may be difficult to wipe off and if the brush is shifted up and down the channel there is furthermore a risk that the biological residue is spread and the channel is contaminated. Furthermore, it is realised that residues on the walls of the channel cannot be flushed out of the channel even by a high flow rate.

Thus, in order to be sure that the channel is properly clean, the cleaning process is carried out for an extensive amount of time.

Accordingly, it is an object of the present invention to provide a cleaning device and a cleaning method for cleaning channels in a medical instrument, such as an endoscope, a ultra sound transducer or the like, which eliminates the risk of leaving residues behind in the channel and to shorten the necessary amount of time for cleaning the channel.

This object is achieved by a device of the initially mentioned kind, wherein the first end may be releasably connected to an outlet flow passage of a cleaning fluid supply means and that the flow passage of the second end is blocked, and that said tubular member is provided with a plurality of fluid openings along at least a section between the first and second ends, so that vaporised cleaning fluid and pressurised gas from the cleaning fluid supply means may be sprayed into the medical instrument channel for flushing the channel.

By the invention there is also provided an apparatus for cleaning a channel in a medical instrument, said apparatus comprising a supply unit comprising a cleaning fluid reservoir, vapour generating means for vaporising of fluid from the cleaning fluid reservoir, gas pressurising means for pressurising gas from a gas source, and control means for controlling the flow of vapour and/or pressurised air to an outlet flow passage; and a device for insertion in a channel of a medical instrument where said device is in flow communication with the outlet flow passage.

Moreover, the invention also concerns a method of cleaning a channel in a medical instrument, said method comprising the steps of: providing a medical instrument, such as an endoscope, having at least one biopsy channel, inserting a flexible elongated tubular member in the channel, and then performing a channel cleaning cycle, which comprises the steps of discharging a vaporised cleaning fluid through a plurality of openings along the elongated tubular device into the channel for releasing any impurities therein, and then discharging pressurised gas through a plurality of openings along the elongated tubular device into the channel for ejecting the particles in the channel.

According to the invention, it is possible to impact the channel inner wall in its entire length with a vapour of cleaning fluid at predetermined temperature and at predetermined pressure, preferably steam from demineralised water, or detergents in liquid form. By running a cleaning cycle where hot vapour, preferably steam, is discharged into the channel and then succeeded by pressurised air for flushing the channel, the channel is cleaned in a quick and efficient manner, as any residue is released from the sides by the steam and then blown out of the channel by the pressurised air. By repeating this cleaning cycle, i.e. altering between discharging vapour and pressurised gas, the channel can be rapidly cleaned or even sterilised if appropriate fluids, such as an ozone gas, is used.

By a method and an apparatus according to the invention, the medical instrument can be washed and cleaned after use not only on the outside but also in the channels, such as the biopsy channels, and thereby preparing the instrument for sterilisation. Furthermore, it is realised that by using appropriate gasses and fluids, the method may also be performed during the sterilisation process succeeding the washing and general cleaning of the instrument.

In a preferred embodiment, the elongated tube member is a flexible tubular member made of Teflon™ material. By using Teflon, such as PTFE (polytetraflouroethylene) or similar flouropolymer materials, the elongated tubular member of the device is provided with a clean chemically inert surface whereby the risk of adhesion of any contamination is prevented. However, it is realised that other materials may be used, such as biologically inert steel or the like.

Preferably, the elongated member is mounted in a connector member at the first end. Hereby, a fluid flow communication between the supply unit and the device may be established as well as the connector may serve as a stop so that only the elongated tubular member can be inserted into the channel.

In a first embodiment, the fluid openings are cut outs in the wall of the tubular member. In particular, the openings are alternately provided in two opposite rows on the wall of the tubular member. By providing the openings in the tubular member in this manner, it is easy to produce the device at relative low costs. Alternatively—or in combination—the fluid openings are holes drilled in the wall of the tubular member. The fluid openings are preferably provided in at least two, preferably radially oppositely situated rows in the wall of the tubular member.

In an embodiment of the invention, the second end is adapted for receiving a detachable brush head. In particular, the second end may be provided with retention means for receiving and retaining the brush head. These retention means could be a splitting in the end section. This could—with or without the brush mounted thereon—also serve the purpose of retaining the elongated tubular member inside the channel during the cleaning operation and prevent the device from being ejected out of the channel when the steam or pressurised gas is released.

In a preferred embodiment of the invention, the cleaning fluid used is demineralised water, and accordingly, the vapour generating means of the supply unit is a steam generator.

Preferably, the gas pressurising means comprising an inlet for receiving ambient air and ambient air is used as the flushing gas in the channel. Preferably, the gas pressure of the generated pressurised gas is between 1 to 3 bars, preferably approx. 2 bars.

The gas pressuring means may also comprise a gas supply, such as an ozone gas source. This could be advantageous for sterilisation purposes.

In the preferred embodiment of the invention, the control means of the apparatus are adapted to providing a cleaning cycle comprising a first interval where vaporised fluid is forwarded to the device and discharged into the channel and a second interval where pressurised gas is forwarded and discharged from the device into the channel. In particular, the first interval is smaller than the second interval, preferably the first interval is between 01-10 seconds, most preferably approx. 1 second, and preferably the second interval is between 20-30 seconds. Hereby, an efficient and gentle cleaning operation may be achieved, as the inner wall of the channel is not heated or impacted to a degree where there is any risk of damaging the channel of the medical instrument neither by the temperature nor by the gas pressure.

In the following, the invention is further explained with reference to the accompanying drawings, in which:

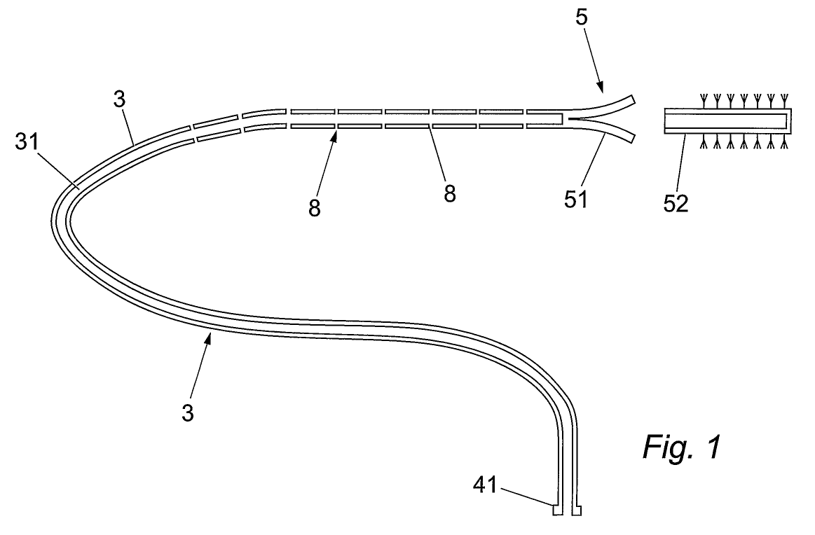

FIG. 1 is a schematic drawing of a device according to the invention,

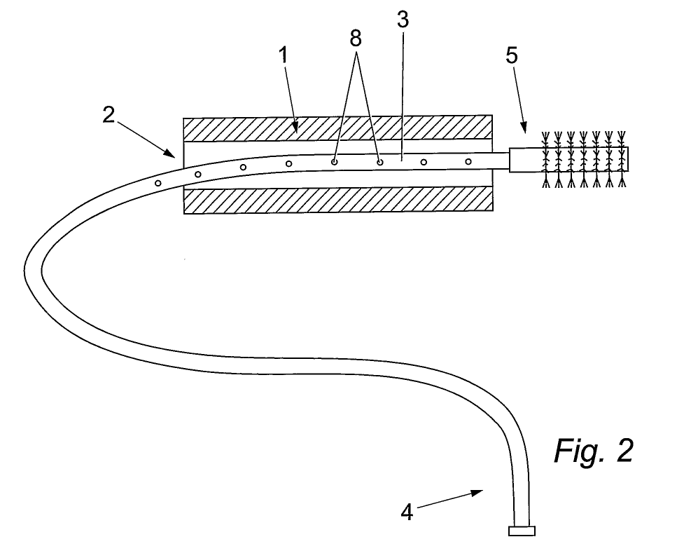

FIG. 2 is a schematic drawing of the device inserted in a channel,

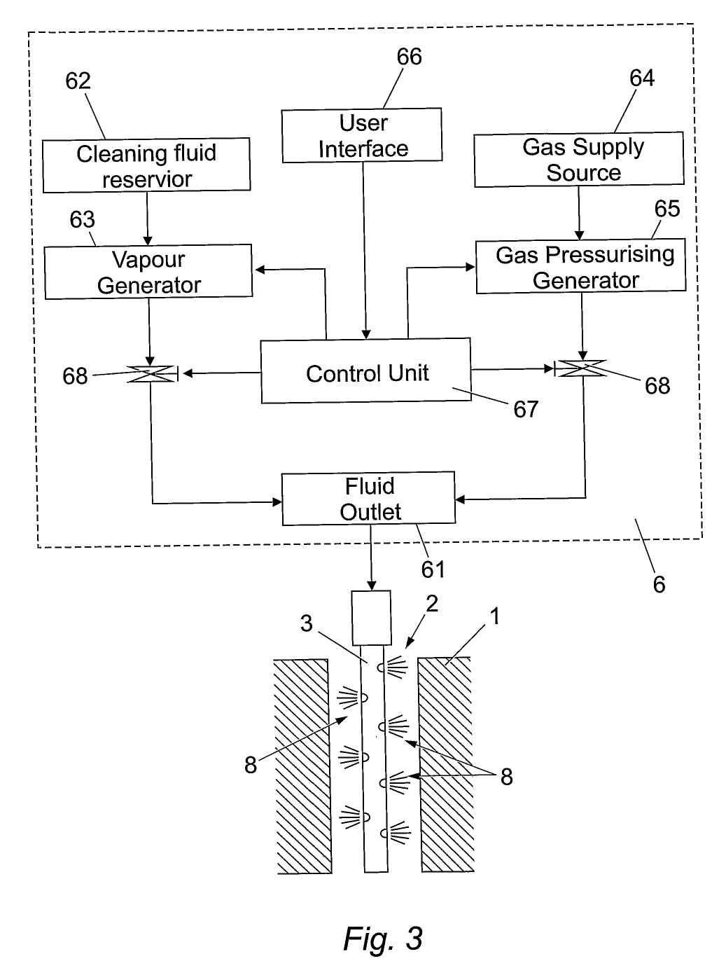

FIG. 3 is a block diagram of a system according to an embodiment of the invention,

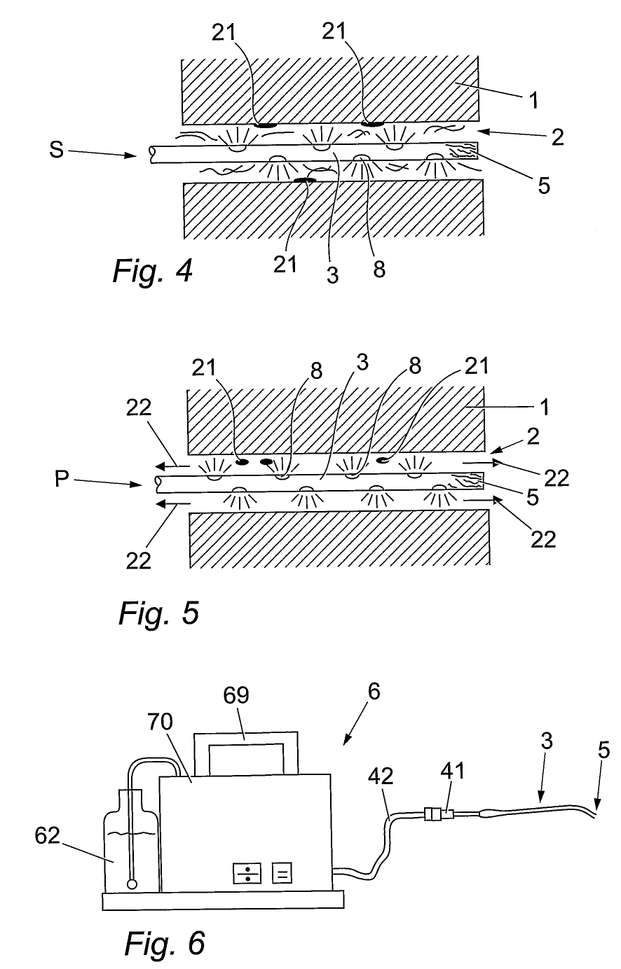

FIG. 4 is a schematic illustration of the first step in a cleaning cycle according to the invention,

FIG. 5 is a schematic illustration of the second step in a cleaning cycle according to the invention, and

FIG. 6 is an illustration of an apparatus according to the invention.

With reference to FIGS. 1 and 2, there is shown a device for cleaning a channel 2 of a medical instrument 1, such as an endoscope, a transducer or the like for medical examination or surgery of internal body tissue of a patient. The device is adapted for being connected to a cleaning fluid supply unit 6 (see FIG. 3 and FIG. 6).

The device comprises a flexible or rigid tubular member 3 having a fluid flow passage 31 for supplying a fluid flow of liquid or a gas to flow from a first end 4 which via a connector head 41 is releasably attached to a fluid outlet 61 of the fluid supply unit 6 to a number of openings 8 provided at least in a section of the tubular member 3, which is terminated at its second, distal end 5, where the flow passage 31 is blocked. The second end 5 may be provided with retaining means 51, such as a split end (as shown in FIG. 1) which is capable of retaining the device inside the channel 2. The retaining means 51 may also serve as a fastener for a brush head 52, whereby the channel may be swept during the insertion of the device in the channel and thereby pushing blocking residue out of the channel during the insertion of the device. This function may be enhanced by provided the device with a brush head 52, but it is realised by the invention that other designs of the distal end 5 will provide the device with the same functionality.

The openings 8 provided in the flexible tubular member 3 are preferably provided in a perforated section ending at the second end 5. The perforated section is preferably provided with a length of at least the length of the channel to be cleaned. Accordingly, a set of devices in different lengths may be provided in order to enable the user to clean channels in medical instruments of different lengths. Similarly, the device may also be provided with different sizes of openings, number of openings and different diameters in order to optimise the device to channels of particular medical instrument designs. Typically, the biopsy channels are between 1.0-5.0 mm in diameter and 100-1200 mm in length. Accordingly, the device is preferably provided with a tubular member of 0.5-4.8 mm in outer diameter and with a perforated section of 90-1190 mm in length.

The openings 8 may be provided as holes opposite each other, such as shown in FIG. 1, e.g. by drilling holes through the tubular member. In another embodiment, the openings 8 may be cut out in the wall of the tubular member 3 with alternating positions with respect to the two sides of the member (see FIG. 3-5).

The method the device is used for cleaning a medical instrument channel 2 may be explained by reference to FIG. 3.

The elongated tubular member 3 of a cleaning device is inserted into a channel 2 of a medical instrument 1, so that the distal end—with or without a brush head—immerged on the other side of the channel 2, whereafter the brush head (if any) is removed. The device is connected at its connector 41 to the fluid outlet 61 of the fluid supply unit 6.

In the fluid supply unit 6, a cleaning fluid reservoir 62 is provided. This reservoir may be a bottle or similar sealed container of demineralised water. The cleaning fluid is pumped (by pumping means not shown) through a vapour generator 63 whereby pressurised steam is generated. This vapour is forwarded through a control valve 68 to the fluid outlet. The control valve 68 is controlled by a control unit 67 which controls the channel cleaning process according to a predetermined cleaning process. A user interface 66 may be provided for selecting the cleaning process or simply to start and stop the process.

In a second supply string, a gas supply source 64 is provided. The gas supply source 64 could be an air intake sucking ambient air into the gas pressurising generator 65 for generating pressurised gas. As an alternative or in supplement to the ambient air, a gas different from ambient air may be used for particular cleaning purposes, e.g. for sterilising the inner space of the channel. The pressurised gas is forwarded from the pressure generator 65 to the fluid outlet 61 via a control valve 68, which is controlled by the control unit 67.

In a cleaning pattern according to a preferred embodiment, the control unit 67 carries out a predetermined amount of cleaning cycles. Each cleaning cycle includes first interval (see FIG. 4) where steam—indicated by arrow S—is released into the tubular member 3 and through the openings 8 and into the inner space of the channel 2 for releasing any residue 21 on the inner wall of the channel. The first interval of the cleaning cycle is followed by a second interval (see FIG. 5) where the steam S is succeeded by a supply of pressurised gas P which is blasted through the openings and into the channel 2. The gas escapes the channel through the channel openings. As the gas flows towards the channel opening (indicated by arrows 22) the impurities 21 released by the steam during the first interval are expelled from the channel 2 by the air blast in the second interval of the cleaning cycle. The pressurised gas may be forwarded through the tubular member 3 with a pressure of 0.5-3 bars.

In a preferred embodiment, the entire cleaning cycle is 30 seconds and the duration of the first interval is approx. 1 second. The steam may be forwarded through the tubular member 3 with a pressure of 0.5-3 bars.

The cleaning cycle may be repeated for about 5 minutes which is found to be sufficient to wash out impurities in a typically sized biopsy channel of an endoscope or the like. However, by the invention it is realised that the duration of the intervals in the cleaning cycle and the amount of cycles performed may vary depending on the actual instrument to be cleaned.

As shown in FIG. 6, the fluid supply unit 6 may be provided as a compact unit which is portable. The components of the unit are accommodated in a housing 70 with the user interface 66 on an exterior side facilitating a user selection of cleaning cycle processes or the like, as well as connecting the unit to an AC voltage supply, e.g. net supply. The housing 69 may be provided with a handle for easy handling. A flexible fluid outlet tube 42 is extending out of the housing 70 and constitutes the fluid outlet 61 of the supply unit 6. The outlet tube 42 is provided with means for connecting the tube 41 to the connector head 41 of the device 3.

Above, the invention is described with reference to one or more preferred embodiments. However, it is realised by the invention that variant may be provided without departing from the scope of the invention as defined in the accompanying claims. As an example, an ozone gas could be used for flushing the channel and thereby sterilising the channel. Accordingly, it is also realised that the apparatus and the method may be used during a sterilisation process of the medical instrument.

1-23. (canceled)

24. A device for cleaning a channel in a medical instrument, said device comprising an elongated tubular member defining a flow passage having a first end and second end, characterised in that the first end may be releasably connected to an outlet flow passage of a cleaning fluid supply means and that the flow passage of the second end is blocked, and that said tubular member is provided with a plurality of fluid openings along at least a section between the first and second ends, so that vaporised cleaning fluid and pressurised gas from the cleaning fluid supply means may be sprayed into the medical instrument channel for flushing the channel.

25. A device according to claim 24, wherein the elongated tube member is a flexible tubular member.

26. A device according to claim 24, wherein the elongated tube member is a tubular member made of a rigid material.

27. A device according to claim 24, wherein the elongated member is mounted in a connector member at the first end.

28. A device according to claim 24, wherein the fluid openings are cut outs in the wall of the tubular member.

29. A device according to claim 24, wherein the fluid openings are holes drilled in the wall of the tubular member.

30. A device according to claim 24, wherein the fluid openings are provided in at least two rows in the wall of the tubular member.

31. A device according to claim 30, wherein the openings are alternately provided in two opposite rows on the wall of the tubular member.

32. A device according to claim 24, wherein the second end is adapted for receiving a detachable brush head.

33. A device according to claim 30, wherein said second end is provided with retention means for receiving and retaining the brush head.

34. An apparatus for cleaning a channel in a medical instrument, said apparatus comprising
a supply unit comprising
a cleaning fluid reservoir,
vapour generating means for vaporising of fluid from the cleaning fluid reservoir,
gas pressurising means for pressurising gas from a gas source, and
control means for controlling the flow of vapour and/or pressurised air to an outlet flow passage; and
a device for insertion in a channel of a medical instrument where said device is in flow communication with the outlet flow passage.

35. An apparatus according to claim 34, wherein the device is a device according to claim 24.

36. An apparatus according to claim 34, wherein the cleaning fluid is demineralised water.

37. An apparatus according to claim 36, wherein the vapour generating means are a steam generator.

38. An apparatus according to claim 34, wherein gas pressurising means comprising an inlet for receiving ambient air.

39. An apparatus according to claim 34, wherein gas pressuring means comprises a gas supply.

40. An apparatus according to claim 34, wherein the control means are adapted to providing a cleaning cycle comprising a first interval where vaporised fluid is forwarded to the device and discharged into the channel and a second interval where pressurised gas is forwarded and discharged from the device into the channel.

41. An apparatus according to claim 40, wherein the first interval is smaller than the second interval.

42. A method of cleaning a channel in a medical instrument, said method comprising the steps of:
providing a medical instrument, such as an endoscope, having at least one biopsy channel,
inserting a flexible elongated tubular member in the channel, and then
performing a channel cleaning cycle, which comprises the steps of
discharging a vaporised cleaning fluid through a plurality of openings along the elongated tubular device into the channel for releasing any impurities therein, and then
discharging pressurised gas through a plurality of openings along the elongated tubular device into the channel for ejecting the particles in the channel.

43. A method according to claim 42, whereby the channel cleaning cycle is repeated a plurality of times.

44. A method according to claim 42, whereby the cleaning cycle includes the vapour discharge for a first time interval and a gas pressure discharge for a second interval.

45. A method according to claim 44, whereby the first interval is smaller than the second interval.

46. A method according to claim 42, whereby the gas pressure is between 1 to 3 bars.

47. A device according to claim 25, wherein the flexible tubular member is made of a fluoropolymer.

48. A device according to claim 26, wherein the tubular member is a made of a metal.

49. A device according to claim 30, wherein the fluid openings are provided in at least two radially oppositely situated rows in the wall of the tubular member.

50. An apparatus according to claim 39, wherein gas pressuring means comprises an ozone gas source.

51. An apparatus according to claim 41, wherein the first interval is between 01-10 seconds and the second interval is between 20-30 seconds.

52. An apparatus according to claim 45, wherein the first interval is approximately 1 second.

53. A method according to claim 45, whereby the first interval is between 01-10 seconds and the second interval is between 20-30 seconds.

54. A method according to claim 53, wherein the first interval is approximately 1 second.

55. A method according to claim 46, whereby the gas pressure is approx. 2 bars.
